(19) United States
(12) Patent Application Publication
(10) Pub. No.: US 2025/0235595 A1
(43) Pub. Date: Jul. 24, 2025

(54) Intravascular sheath

(71) Applicant: Inspire M.D Ltd., Tel Aviv (IL)

(72) Inventors: Boris Kadtsyn, Yad Rambam (IL); Shmuel Vered, Haifa (IL); Kurt Garner, Grand Rapids, MI (US); Jose Ignacio Leal Lorenzo, Madrid (ES)

(21) Appl. No.: 18/985,163

(22) Filed: Dec. 18, 2024

Related U.S. Application Data
(60) Provisional application No. 63/622,106, filed on Jan. 18, 2024.

(51) Int. Cl. A61M 1/36; A61M 25/00; A61M 25/01; A61M 25/02; A61M 25/06

(52) U.S. Cl. CPC A61M 1/3655; A61M 25/0097; A61M 25/0108; A61M 25/02; A61M 25/0662; A61M 2025/024; A61M 2025/0286

(57) ABSTRACT

Surgical apparatus includes a tubular sheath having a lumen passing longitudinally therethrough. The sheath includes a flexible distal section, configured for percutaneous insertion into an artery of a patient and including radiopaque gradations to indicate a depth of penetration of the sheath into the artery and having a distal port communicating with the lumen, and a rigid proximal section. A hub connected to a proximal end of the proximal section includes first and second proximal ports communicating with the lumen, such that the first proximal port is coaxial with the sheath and the second proximal port is angled relative to the sheath.

### CROSS-REFERENCE TO RELATED APPLICATION

This application claims the benefit of U.S. Provisional Patent Application 63/622,106, filed Jan. 18, 2024, which is incorporated herein by reference.

### FIELD

The present invention relates generally to medical devices, and particularly to intravascular sheaths and accessories.

### BACKGROUND

Some medical procedures, such as treatment of arterial stenoses and strokes, require a vascular surgeon or neurosurgeon to access a patient's carotid arteries. A favored approach for this purpose involves insertion of a thin sheath into the common carotid artery through a small incision in the patient's neck. Specialized tools can then be inserted through the sheath, for example to dilate stenoses or to remove clots that are occluding cerebral blood vessels. Systems and methods for this purpose are described, for example in U.S. Patent Application Publication 2021/0307945.

Some surgical procedures induce reverse blood flow in the carotid artery to protect the brain from emboli that may be released during the procedure. For this purpose, as in U.S. explained Patent Application Publication 2021/0307945, an arterial access cannula is connected to a venous cannula in order to establish a retrograde flow from the internal carotid artery into the venous system, for example into the jugular or femoral vein. Flow in the common carotid artery can be temporarily occluded by various means. After such reverse or retrograde flow is established, the surgical procedure can be performed with a reduced risk of emboli entering the cerebral vasculature.

### SUMMARY

Embodiments of the present invention that are described hereinbelow provide improved devices and methods for treatment of the vascular system.

There is therefore provided, in accordance with an embodiment of the invention, surgical apparatus, including a tubular sheath having a lumen passing longitudinally therethrough and including a flexible distal section, configured for percutaneous insertion into an artery of a patient and including radiopaque gradations to indicate a depth of penetration of the sheath into the artery and having a distal port communicating with the lumen, and a rigid proximal section. A hub is connected to a proximal end of the proximal section and includes first and second proximal ports communicating with the lumen, such that the first proximal port is coaxial with the sheath and the second proximal port is angled relative to the sheath.

In a disclosed embodiment, the flexible distal section has a preformed angular bend.

Additionally or alternatively, the apparatus includes a dilator rod configured for insertion into the lumen through the first proximal port and terminating in a distal tip, which protrudes through the distal port into the artery when the dilator rod is fully inserted into the lumen.

In a disclosed embodiment, the apparatus includes a fixation clip, which includes a collar configured to be fitted over the proximal section of the sheath in a desired location and to grasp the sheath at the desired location and one or more eyelets fixed to the collar and configured for passage of a suture therethrough so as to secure the fixation clip to skin of the patient.

In a further embodiment, the apparatus includes a shunt tube for connection between the second proximal port and a venous sheath for insertion into a vein of the patient so as to convey a retrograde flow of blood from the artery to the vein through the sheath and the shunt tube.

In a disclosed embodiment, the first proximal port includes a luer fitting configured for connection of a valve thereto.

In some embodiments, the radiopaque gradations include radiopaque bands extending circumferentially around the distal section at predefined intervals. Additionally or alternatively, the radiopaque gradations include numbers.

In an optional embodiment, the apparatus includes a stopper, is which configured to slide longitudinally along the distal section of the sheath to a desired location and to grasp the sheath at the desired location so as to limit a depth of penetration of the sheath into the artery.

There is also provided, in accordance with an embodiment of the invention, surgical apparatus, including a tubular sheath having a lumen passing therethrough and including a distal section configured for percutaneous insertion into an artery of a patient and a proximal section connected to the distal section. At least one fixation clip includes a collar configured to be fitted over the proximal section of the sheath in a desired location and to grasp the sheath at the desired location and one or more eyelets fixed to the collar and configured for passage of a suture therethrough so as to secure the fixation clip to skin of the patient.

In a disclosed embodiment, the at least one fixation clip includes multiple fixation clips, which are configured to be fitted over the proximal section of the sheath at different, respective locations.

Additionally or alternatively, the collar has a frictional inner surface to grip an outer surface of the sheath.

There is additionally provided, in accordance with an embodiment of the invention, a surgical method, which includes inserting a flexible distal section of a tubular sheath percutaneously into an artery of a patient. The sheath has a lumen passing longitudinally therethrough, and the flexible distal section includes radiopaque gradations. A depth of penetration of the sheath into the artery is controlled by observing the radiopaque gradations in a fluoroscopic image.

In some embodiments, the sheath includes a hub connected to a proximal end of the sheath, the hub including first and second proximal ports communicating with the lumen, such that the first proximal port is coaxial with the sheath and the second proximal port is angled relative to the sheath. In a disclosed embodiment, the method includes inserting a dilator rod into the lumen through the first proximal port so that a distal tip of the dilator rod protrudes through a distal port of the sheath into the artery when the dilator rod is fully inserted into the lumen. Additionally or alternatively, the method includes inserting a venous sheath into a vein of the patient and connecting a shunt tube between the second proximal port of the hub and the venous sheath so as to convey a retrograde flow of blood from the artery to the vein through the sheaths and the shunt tube.

There is further provided, in accordance with an embodiment of the invention, a surgical method, which includes inserting a distal section of a tubular sheath percutaneously into an artery of a patient, the sheath having a lumen passing therethrough and including a proximal section connected to the distal section. At least one fixation clip is fitted over the proximal section of the sheath in a desired location so that the clip grasps the sheath at the desired location. The fixation clip is secured to skin of the patient using a suture passed through one or more eyelets on the fixation clip.

The present invention will be more fully understood from the following detailed description of the embodiments thereof, taken together with the drawings in which:

### BRIEF DESCRIPTION OF THE DRAWINGS

FIG. 1 is a schematic pictorial illustration showing insertion of a sheath into a carotid artery, in accordance with an embodiment of the invention;

FIG. 2 is a schematic side view of an arterial sheath, in accordance with an embodiment of the invention;

FIG. 3 is a schematic side view of a dilator rod for insertion through the sheath of FIG. 2, in accordance with an embodiment of the invention;

FIG. 4A is a schematic pictorial view of a distal part of an arterial sheath, in accordance with an embodiment of the invention;

FIG. 4B is a schematic pictorial view of a distal part of an arterial sheath, in accordance with another embodiment of the invention;

FIG. 5 is a schematic pictorial view of a fixation clip for a vascular sheath, in accordance with an embodiment of the invention;

FIG. 6 is a schematic side view of an adjustable stopper fitted over an arterial sheath, in accordance with an embodiment of the invention;

FIG. 7 is a schematic side view of a venous sheath, in accordance with an embodiment of the invention; and

FIG. 8 is a schematic frontal view of a kit for reversal of arterial blood flow, in accordance with an embodiment of the invention.

### DETAILED DESCRIPTION

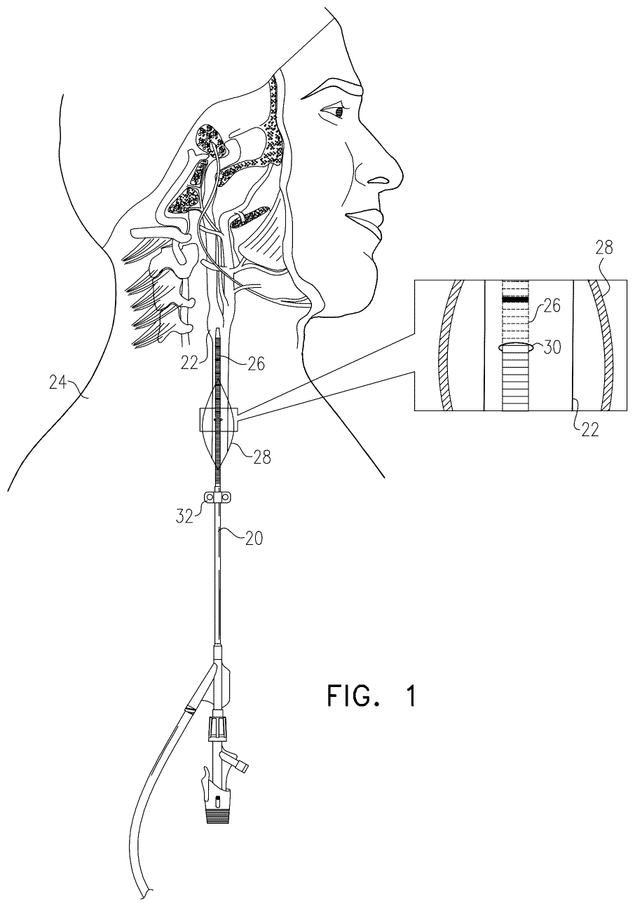

FIG. 1 is a schematic pictorial illustration showing insertion of a sheath 20 into a carotid artery 22 of a patient 24, in accordance with an embodiment of the invention. For this purpose, a surgeon makes a small incision 28 in the patient's neck and then inserts a distal section 26 of sheath 20 through a small puncture 30 in the artery. After insertion of sheath 20 to the desired depth within artery 22, the surgeon secures the sheath in place by suturing a fixation clip 32 to the patient's skin.

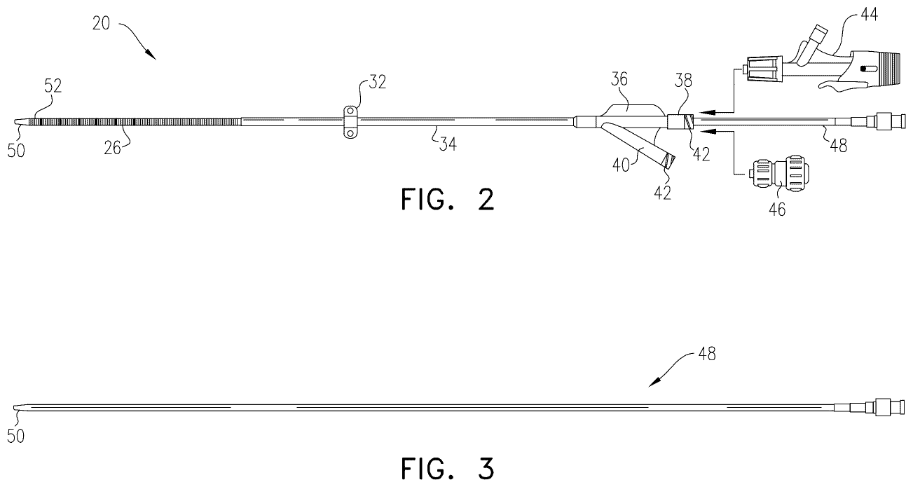

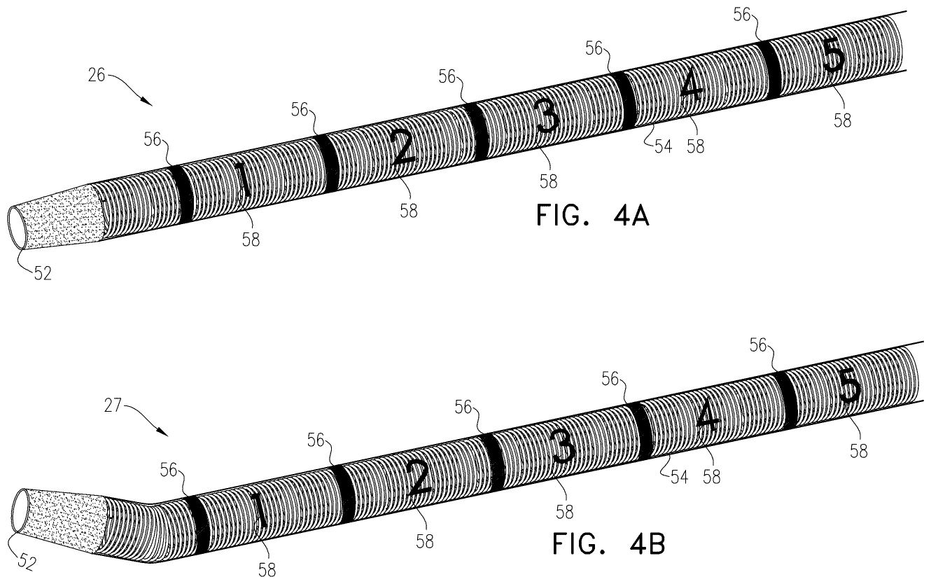

FIG. 2 is a schematic side view of arterial sheath 20, in accordance with an embodiment of the invention. Sheath 20 has a tubular structure, with an internal lumen passing longitudinally through the sheath. Distal section 26, which is flexible (as shown in detail in FIG. 4A), is connected at its proximal end to a rigid proximal section 34. A hub 36 is connected to the proximal end of proximal section 34. Hub 36 comprises proximal ports 38 and 40 communicating with the lumen in sheath 20. Proximal port 38 is coaxial with sheath 20, while proximal port 40 is angled relative to the sheath.

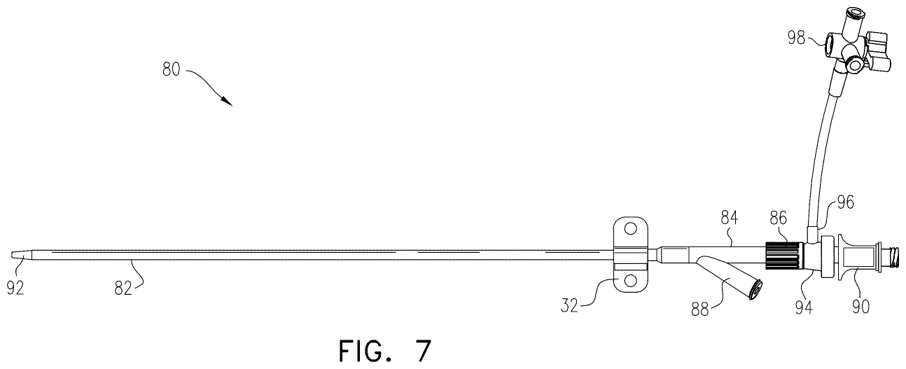

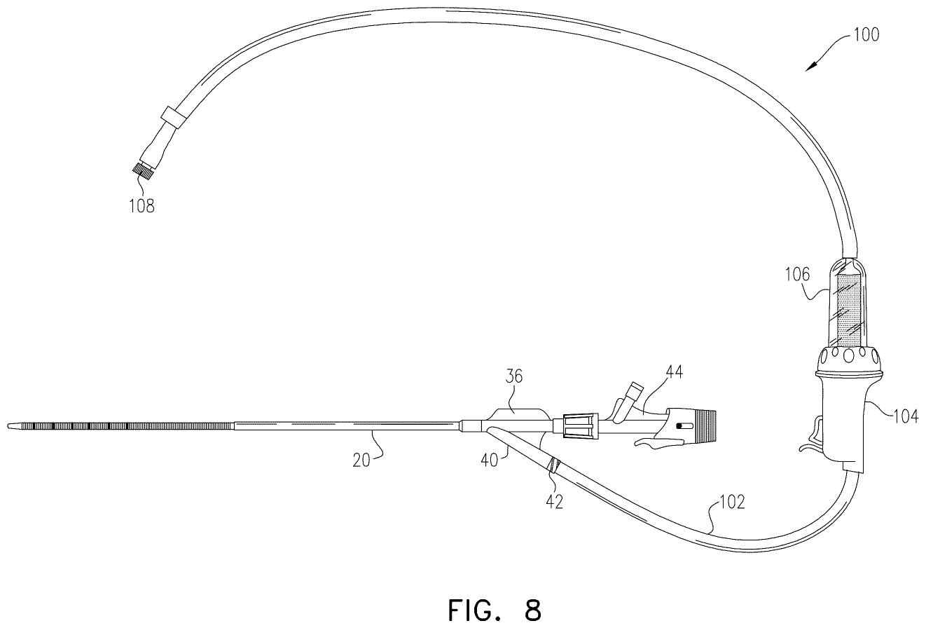

Ports 38 and 40 comprise fittings 42, such as luer fittings, which enable the surgeon or other user to attach different sorts of valves and fluid flow components to the ports. The pictured embodiment shows two types of homeostasis valves that can be attached to port 38 in this manner: a Y-valve 44 and a Tuohy-Borst adapter 46 Alternatively, other types of valves that are known in the art may be attached to port 38, such as a “copilot” valve or a silicone anti-reflux valve (for example as shown in FIG. 7). Port 40 may be connected to an arterial/venous shunt, as shown in FIG. 8.

FIG. 3 is a schematic side view of a dilator rod 48 for insertion through sheath 20, in accordance with an embodiment of the invention. As shown in FIG. 2, dilator rod 48 is inserted into the lumen in sheath 20 through proximal port 38. Dilator rod 48 terminates in a distal tip 50, which protrudes through a distal port 52 of sheath 20 into artery 22 when the dilator rod is fully inserted into the lumen. Distal tip 50 may be symmetrical about the longitudinal axis of dilator rod 48 as shown in FIG. 3, or it may alternatively be asymmetrical, for example flattened on one side of the longitudinal axis and rounded on the other. Although dilator rod 48 is shown in FIG. 2 as being inserted directly through port 38, in surgical practice the dilator rod is more commonly inserted through a suitable homeostasis valve, such as valve 44, which is connected to port 38.

FIG. 4A is a schematic pictorial view of distal section 26 of arterial sheath 20, in accordance with n embodiment of the invention. Distal section 26 comprises a flexible, resilient structure 54, such as a helical coil, which terminates at distal port 52. Distal section 26 is typically a few centimeters in length and may be connected to rigid proximal section 34 (FIG. 2) by a transition section of intermediate flexibility.

Distal section 26 is made of a radiopaque material and includes radiopaque gradations 56, 58, which can be seen in fluoroscopic images during the insertion procedure and indicate a depth of penetration of sheath 20 into the artery. In the pictured example, gradations 56 comprise radiopaque bands extending circumferentially around the distal section at predefined intervals. Gradations 58 comprise numbers, which give a quantitative measure of the depth of penetration. Alternatively, distal section 26 may comprise only one type of gradations 56 or 58, or it may comprise radiopaque gradations having other geometrical forms. During insertion of distal section 26 into the artery, it is advantageous to rotate the distal section so that gradations 58 face outward, toward the patient's body surface, so that the gradations can be seen clearly in fluoroscopic images.

FIG. 4B is a schematic pictorial view of a distal section 27 of an arterial sheath, in accordance with an alternative embodiment of the invention. In this embodiment, the tip of distal section 27 is bent, for example by about 30O. This sort of bent shape is useful in avoiding accidental dissection of the carotid artery during the procedure. Distal section 27 may be preformed with the desired angular bend, or it may alternatively be made of a malleable material that allows the surgeon to set the curve angle as desired. The bend in distal section 27 may advantageously be oriented toward the side of the sheath on which gradations 58 are marked, so that when the sheath is properly rotated, the gradations appear clearly in fluoroscopic images.

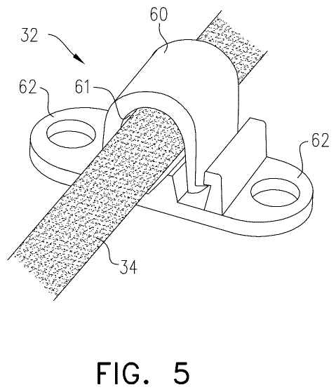

FIG. 5 is a schematic pictorial view showing details of fixation clip 32, in accordance with an embodiment of the invention. Clip 32 comprises a collar 60, which can be fitted over proximal section 34 of sheath 20 at any desired location. Collar 60 clips over and grasps the sheath at the desired location. One or more eyelets 62 are fixed to collar 60. The surgeon passes one or more sutures through eyelets 62 and stitches the sutures to the patient's skin in order to secure fixation clip 32 in place and thus prevent further movement of sheath 20 until the surgical procedure has been completed. For greater security, two or more fixation clips 32 can be fitted over proximal section 34 at different locations and sutured to the patient's skin.

To ensure that sheath 20 remains stationary during the procedure, collar 60 may have a frictional inner surface 61, to grip the outer surface of the sheath. For example, inner surface 61 may include protruding, flexible ribs. Alternatively or additionally, inner surface 61 may be roughened or may even have a sticky coating.

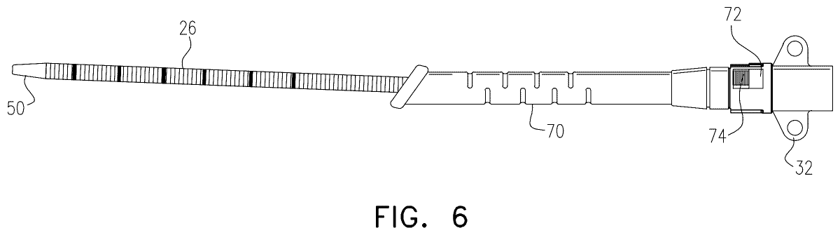

FIG. 6 is a schematic side view of an adjustable stopper 70, which is fitted over sheath 20, in accordance with an alternative embodiment of the invention. The use of such a stopper is optional, although some surgeons may feel that it enhances the safety of the insertion procedure. Stopper 70 can be slid longitudinally along distal section 26 of sheath 20 to a desired location. A locking mechanism 72 clips over a stop 74 to grasp the sheath at the desired location. Stopper 70 may have a frictional inner surface, as in clip 32, to prevent movement of the sheath after locking. The distal end of stopper 70 then limits the depth of penetration of the sheath into the artery. Although only one stop 74 is visible in FIG. 6, multiple stops can be disposed along the length of proximal section 34 to enable the longitudinal location of stopper 70 to be adjusted.

FIG. 7 is a schematic side view of a venous sheath 80, in accordance with another embodiment of the invention. Sheath 80 (like sheath 20) has a tubular structure, with a flexible distal section 82 and an internal lumen passing longitudinally through the sheath. A hub 84 at the proximal end of sheath 80 comprises proximal ports 86 and 88 communicating with the lumen in the sheath. Ports 86 and 88 comprise fittings, such as luer fittings, which enable the surgeon or other user to attach different sorts of valves and fluid flow components to the ports.

In the pictured example, a silicone anti-reflux valve 94 is connected to port 86. Valve 94 has a sidearm 96, which connects to a fluid control hub 98. A dilator rod 90 is inserted through via valve 94 through sheath 80, so that a distal time 92 of the dilator rod protrudes through the distal port of distal section 82.

FIG. 8 is a schematic frontal view of a kit 100 for reversal of arterial blood flow, in accordance with an embodiment of the invention. Kit 100 comprises sheath 20 and associated components (such as hub 36, valve 44, and dilator rod 48) that were described above. Kit 100 also comprises a shunt tube 102, which is connected to proximal port 40 and connects at its distal end 108 to venous sheath 80 (FIG. 7) for insertion into a vein of the patient. Shunt tube 102 thus conveys a retrograde flow of blood from the artery to the vein through sheath 20 from artery 22 (FIG. 1) to the target vein, such as to the jugular or femoral vein.

Optionally, a flow control valve 104 and a filter 106 are connected in line with shunt tube 102. Further details of these sorts of components for use in a retrograde flow system are described, for example, in U.S. Pat. No. 11,844,893 and in PCT International Publication WO 2022/201081, whose disclosures are incorporated herein by reference.

Although the embodiments described above refer specifically to treatments performed on and/or through the carotid arteries, the devices and techniques described above may alternatively be applied, mutatis mutandis, in other blood vessels. Furthermore, although the components and accessories of sheath 20 and kit 70 are shown together in a particularly useful and advantageous configuration, the various elements of the embodiments described above may alternatively be used individually or in other combinations.

Thus, the embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

The invention claimed is:

1. Surgical apparatus, comprising:
a tubular sheath having a lumen passing longitudinally therethrough and comprising:
a flexible distal section configured for percutaneous insertion into an artery of a patient and comprising radiopaque gradations to indicate a depth of penetration of the sheath into the artery and having a distal port communicating with the lumen; and
a rigid proximal section; and
a hub connected to a proximal end of the proximal section and comprising first and second proximal ports communicating with the lumen, such that the first proximal port is coaxial with the sheath and the second proximal port is angled relative to the sheath.

2. The apparatus according to claim 1, wherein the flexible distal section has a preformed angular bend.

3. The apparatus according to claim 1, and comprising a dilator rod configured for insertion into the lumen through the first proximal port and terminating in a distal tip, which protrudes through the distal port into the artery when the dilator rod is fully inserted into the lumen.

4. The apparatus according to claim 1, and comprising a fixation clip, which comprises:
a collar configured to be fitted over the proximal section of the sheath in a desired location and to grasp the sheath at the desired location; and
one or more eyelets fixed to the collar and configured for passage of a suture therethrough so as to secure the fixation clip to skin of the patient.

5. The apparatus according to claim 1, and comprising a shunt tube for connection between the second proximal port and a venous sheath for insertion into a vein of the patient so as to convey a retrograde flow of blood from the artery to the vein through the sheath and the shunt tube.

6. The apparatus according to claim 1, wherein the first proximal port comprises a luer fitting configured for connection of a valve thereto.

7. The apparatus according to claim 1, wherein the radiopaque gradations comprise radiopaque bands extending circumferentially around the distal section at predefined intervals.

8. The apparatus according to claim 1, wherein the radiopaque gradations comprise numbers.

9. The apparatus according to claim 1, and comprising a stopper, which is configured to slide longitudinally along the distal section of the sheath to a desired location and to grasp the sheath at the desired location so as to limit a depth of penetration of the sheath into the artery.

10. Surgical apparatus, comprising:
a tubular sheath having a lumen passing therethrough and comprising a distal section configured for percutaneous insertion into an artery of a patient and a proximal section connected to the distal section; and
at least one fixation clip, which comprises:
a collar configured to be fitted over the proximal section of the sheath in a desired location and to grasp the sheath at the desired location; and
one or more eyelets fixed to the collar and configured for passage of a suture therethrough so as to secure the fixation clip to skin of the patient.

11. The apparatus according to claim 10, wherein the at least one fixation clip comprises multiple fixation clips, which are configured to be fitted over the proximal section of the sheath at different, respective locations.

12. The apparatus according to claim 11, wherein the collar has a frictional inner surface to grip an outer surface of the sheath.

13. A surgical method, comprising:
inserting a flexible distal section of a tubular sheath percutaneously into an artery of a patient, the sheath having a lumen passing longitudinally therethrough, and the flexible distal section comprising radiopaque gradations; and
controlling a depth of penetration of the sheath into the artery by observing the radiopaque gradations in a fluoroscopic image.

14. The method according to claim 13, wherein the sheath comprises a hub connected to a proximal end of the sheath, the hub comprising first and second proximal ports communicating with the lumen, such that the first proximal port is coaxial with the sheath and the second proximal port is angled relative to the sheath.

15. The method according to claim 14, and comprising inserting a dilator rod into the lumen through the first proximal port so that a distal tip of the dilator rod protrudes through a distal port of the sheath into the artery when the dilator rod is fully inserted into the lumen.

16. The method according to claim 14, and comprising:
inserting a venous sheath into a vein of the patient; and
connecting a shunt tube between the second proximal port of the hub and the venous sheath so as to convey a retrograde flow of blood from the artery to the vein through the sheaths and the shunt tube.

17. The method according to claim 14, wherein the first proximal port comprises a luer fitting configured for connection of a valve thereto.

18. The method according to claim 13, and comprising fitting a fixation clip over a proximal section of the sheath in a desired location so that the clip grasps the sheath at the desired location, and securing the fixation clip to skin of the patient using a suture passed through one or more eyelets on the fixation clip.

19. The method according to claim 13, wherein the radiopaque gradations comprise radiopaque bands extending circumferentially around the distal section at predefined intervals.

20. The method according to claim 13, wherein the radiopaque gradations comprise numbers.

21. The method according to claim 13, and comprising sliding a stopper longitudinally along the distal section of the sheath to a desired location and so that the stopper grasps the sheath at the desired location and limits a depth of penetration of the sheath into the artery.

22. A surgical method, comprising:
inserting a distal section of a tubular sheath percutaneously into an artery of a patient, the sheath having a lumen passing therethrough and comprising a proximal section connected to the distal section;
fitting at least one fixation clip over the proximal section of the sheath in a desired location so that the clip grasps the sheath at the desired location; and
securing the fixation clip to skin of the patient using a suture passed through one or more eyelets on the fixation clip.

23. The method according to claim 22, wherein fitting the at least one fixation clip comprises fitting multiple fixation clips over the proximal section of the sheath at different, respective locations, for securing to the skin at the respective locations.
